(19) United States
(12) Patent Application Publication
(10) Pub. No.: US 2012/0016112 A1
(43) Pub. Date: Jan. 19, 2012

(54) PROCESSING OF MACRONUTRIENTS

(75) Inventors: Raymond-David Pridmore, St-Sulpice (CH); Fabrizio Arigoni, Geneve (CH); Francoise Maynard, Speigel Bel Bern (CH); Isabelle Bureau-Franz, Epalinges (CH)

(73) Assignee: Nestec S.A., Vevey (CH)

(21) Appl. No.: 13/140,731

(22) PCT Filed: Dec. 11, 2009
(86) PCT No.: PCT/EP2009/066903

§ 371 Date: Jun. 17, 2011

(30) Foreign Application Priority Data
Jan. 6, 2009 (EP) 09150113.0

(51) Int. Cl. C07K 14/00; C12P 21/00

(52) U.S. Cl. 530/407; 435/68.1

(57) ABSTRACT

The present invention generally relates to edible compositions and methods to produce them. In particular, the present invention relates to the enzymatic modulation of macron utrients and to food compositions containing such modulated macronutrients. One embodiment of the present invention is a method for modulating macronutrients comprising the steps of producing at least one synthetic gene coding for at least one enzyme or a functional part thereof capable of modulating macronutrients, expressing the at least one enzyme or a functional part thereof, and bringing the macronutrients into contact with the at least one enzyme or a functional part thereof exhibiting the enzymatic activity.

The present invention generally relates to edible compositions and to methods to produce them. In particular, the present invention relates to the enzymatic modulation of macronutrients and to food compositions containing such modulated macronutrients.

Food products typically contain nutrients. Nutrients needed in relatively large quantities are called macronutrients. Typical macronutrients that are generally contained in food products are proteins, carbohydrates and/or lipids. These are often provided in the form of at least one protein source, a carbohydrate source, and/or a lipid source.

While these macronutrient sources are generally used in the form that nature provides, there are cases where it might be preferred to add a macronutrient source in a modified form to a food composition.

For example, in subjects with compromised functioning of the gastro-intestinal tract it is preferred, if the subject ingests a diet with short peptide chains to facilitate absorption and food tolerance. For example, nutritional compositions with short peptide chains, such as Peptamen®, has been shown to reduce the incidence of diarrhoea to 0% compared to 40% in ICU patients receiving an intact protein formula (Meredith et al J Trauma 1990; 30:825-829).

Such a nutritional composition is in particular appropriate for metabolically stressed children, those with compromised gastro-intestinal function and those with challenging feeding issues.

Peptamen® contains as a protein source peptides from hydrolysed whey protein, which provide an easily absorbed and well utilised source of nitrogen. These whey derived peptides are even better absorbed than free amino acids.

Hydrolysed proteins may also be used by subjects that suffer from allergic disorders. Food allergies, of which the first to occur in life is cows' milk allergy, are caused, in most cases, by a reaction to the proteins in the food. In the early years of life the immune system is still developing and may fail to develop tolerance to dietary antigens (this may also be described as insufficient induction of oral tolerance). The result is that the baby or child or young animal mounts an exaggerated immune response to the dietary protein and develops an allergic response to it. Food allergies may affect not only humans but also other mammals such as dogs and cats. Usually, food hypersensitivity appears just after a susceptible baby, child or young animal first encounters a new food containing potential allergens. Apart from its mother's milk, the first dietary proteins generally encountered by human babies at least are cows' milk proteins and, as noted above, cows' milk allergy is the most common food allergy in human babies. It is generally accepted that babies with established cows' milk allergy have an increased risk of developing atopic diseases and allergies to other dietary proteins such as egg and cereal proteins but even those babies who have successfully developed oral tolerance to cows' milk proteins may subsequently develop allergies to other dietary proteins such as egg and cereal proteins when these are introduced into the diet at weaning. These allergies may manifest themselves clinically as atopic diseases such as atopic dermatitis, eczema and asthma. From a dietary point of view there are two ways to treat an established allergy—either foods containing the allergen must be avoided altogether, or the foods must be treated to decrease their allergenic potential, for example by extensive hydrolysis. Infant formulas containing extensively hydrolysed cows' milk proteins (peptides consisting of not more than five amino acids) are manufactured for this latter purpose. Similarly it has already been proposed, in U.S. Pat. No. 6,403,142 for example, to prepare petfoods with reduced allergenicity for companion animals where it is suspected that the animal has developed a food allergy.

Partially hydrolysed proteins may also be used to induce oral tolerance. Products have been devised which help to reduce the risk of developing the allergy in the first place, particularly for children thought to be at risk of the same (that is, children having at least one close family member who suffers from an allergy). One example of such products is the infant formulas based on partially hydrolysed whey proteins sold under the trade marks NAN HA1 and NAN HA2. These products have been demonstrated to actively induce oral tolerance to cows' milk proteins. Fritsché et al. (J. Allergy Clin. Immunol, Vol 100, No. 2, pages 266-273, 1997) have shown using animal models that enzymatic hydrolysates of cow's milk proteins with a degree of hydrolysis of 18% were able to induce oral tolerance to intact cow's milk proteins whereas hydrolysates with a degree of hydrolysis of 28% were not. Results of these experiments showed that preventive feeding of rats with such a moderately hydrolysed cow's milk formula, whose allergenicity had been reduced over 100 times as compared to a standard formula, suppressed specific IgE and mediator release from intestinal mast cells, both parameters of an immediate type allergic reaction. This work demonstrated that for cows' milk proteins it is possible to define a degree of enzymatic hydrolysis whereby the capacity of the peptides to induce oral tolerance is maintained whilst their allergenicity is substantially reduced.

Typically, macronutrient sources are modified in the food industry today by the use of enzymes that are obtained from natural sources.

For example, whey protein may be hydrolysed using porcine or bovine trypsin and/or chymotrypsin.

Several religions might, however, not allow the use of enzymes obtained from bovine and/or porcine species. Halal foods are one example.

The Codex Alimentarius Commission has issued general guidelines for the use of the term “Halal” (CAC/GL 24-1997). The Codex General Guidelines for the Use of the Term “Halal” were adopted by the Codex Alimentarius Commission at its 22nd Session, 1997. They have been sent to all Member Nations and Associate Members of FAO and WHO as an advisory text and it is for individual governments to decide what use they wish to make of the Guidelines.

The Codex Alimentarius Commission accepts that there may be minor differences in opinion in the interpretation of lawful and unlawful animals and in the slaughter act, according to the different Islamic Schools of Thought. As such, these general guidelines are subjected to the interpretation of the appropriate authorities of the importing countries.

Halal food means food permitted under the Islamic Law.

According to the The Codex Alimentarius Commission the term “halal” may be used for foods which are considered lawful. Under the Islamic Law, all sources of food are lawful except for example the following sources, including their products and derivatives which are considered unlawful: pigs and boars, dogs, snakes and monkeys, carnivorous animals with claws and fangs such as lions, tigers, bears and other similar animals, birds of prey with claws such as eagles, vultures, and other similar birds, pests such as rats, centipedes, scorpions and other similar animals, animals forbidden to be killed in Islam i.e., ants, bees and woodpecker birds, animals which are considered repulsive generally like lice, flies, maggots and other similar animals, animals that live both on land and in water such as frogs, crocodiles and other similar animals, mules and domestic donkeys, all poisonous and hazardous aquatic animals, any other animals not slaughtered according to Islamic Law, blood.

Consequently, for example, porcine trypsin and chymotrypsin, which are typically used to hydrolyse whey protein in the food industry today, may—for some people—not be used for the production of whey protein hydrolysate which shall qualify as halal food.

Obtaining trypsin and chymotrypsin with biotechnological methods from porcine DNA might equally not be considered acceptable by some people, since the source DNA used for this procedure is porcine DNA.

Based on the above it would be desirable to have available a method for modulating macronutrients for food applications which is also in accordance with the requirements of specific religious groups. It would also be desirable to have available a food composition comprising modulated macronutrients, which would be acceptable for consumption by religious groups, for example by qualifying as halal food.

Developing a single method and food composition as described above that is in accordance with the requirements of several religions and at the same time simple and efficient to utilize in an industrial scale, would have the advantage that a single food composition could be sold to believers in religions with food restrictions and to other consumers. At the same time the risk that the consumer accidentally uses the wrong product would be omitted. Having a single product suitable for everybody would also simplify the logistics involved in the production process. Finally, having available such a single method would also contribute to a further improved environmental friendliness.

Consequently, it was the object of the present invention to develop a method for modulating macronutrients, for example for food applications, and a food composition comprising them, which is also in accordance with religious requirements that—for example—do not allow the consumption of parts of certain animals.

The present inventors were surprised to see that they could achieve these objects by a method in accordance with claim 1 and a product in accordance with claim 14.

The present inventors used an enzyme which was produced from a synthetic gene. The enzyme may have the identical amino acid sequence as the enzyme from animal source. At the same time the synthetic gene may also have a DNA sequence that is different from the DNA from animal sources. This way, no mammalian DNA or mammalian material was used to produce the enzyme, but the enzyme remains to have essentially the same amino acid sequence as if it was obtained from animal sources.

The present inventors have produced trypsin from a synthetic gene that expresses an enzyme with the same protein sequence as porcine trypsin and have compared it to trypsin obtained from an animal source.

The present inventors also have produced chymotrypsin from a synthetic gene that expresses an enzyme with the same protein sequence as porcine chymotrypsin and have compared it to chymotrypsin obtained from an animal source.

The functionality of porcine trypsin and chymotrypsin compared to trypsin and chymotrypsin obtained from synthetic genes was found to be virtually indistinguishable.

Consequently the present invention relates to a method for modulating macronutrients comprising the steps of producing at least one synthetic gene coding for at least one enzyme or a functional part thereof capable of modulating macronutrients, expressing the at least one enzyme or a functional part thereof, optionally activating the at least one enzyme or a functional part thereof so that it exhibits enzymatic activity, and bringing the macronutrients into contact with the at least one enzyme or a functional part thereof exhibiting the enzymatic activity.

For example, the present invention concerns a method for modulating macronutrients comprising the steps of producing at least one synthetic gene coding for at least one enzyme or a functional part thereof capable of modulating macronutrients, cloning of this synthesized gene into a micro-organism capable of expressing this gene, cultivating the micro-organism in a culture and expressing the enzyme or a functional part thereof, and bringing the macronutrient into contact with the culture of the micro-organism or a fraction thereof exhibiting the enzymatic activity.

Macronutrients are those nutrients that humans consume in the largest quantities and comprise for example carbohydrates, proteins, and fats.

Modulating macronutrients means altering their chemical structure, for example by hydrolysing and/or rearranging bonds, by modulating the stereochemistry of a macronutrient, and/or by adding atoms or groups of atoms to the macronutrient.

In one preferred embodiment of the present invention the macronutrients are hydrolysed. In the case of carbohydrates this may result in sugars with a shorter chain length. For example polysaccharides may be transformed to oligosaccharides. In general, sugars with a shorter chain length are easier to absorb and will allow generating energy faster and might have functional properties such as prebiotic and anti-infectional properties. In the case of proteins, shorter peptides are generated, which will have for example the advantages described above, other nutritional properties, or can exhibit taste active properties. The hydrolysis of fats will liberate the fatty acids which then also can be absorbed faster by the human body or structured lipids with nutritional benefits might be generated.

Alternatively, it might also be desired to increase the size of the macronutrients, for example in order to provide foods that can provide the body with energy during extended time spans. Short oligosaccharides or mono-saccharides may be ligated, branched or elongated to form sugars with a longer or branched chain length. Equally, free fatty acids may be added, for example to glycerol mono- or diesters, to increase their storage stability or to produce structured lipids with specific fatty acids in sn-1, sn-2 or sn-3 position. Finally functional groups may be added to proteins or peptides, for example to modify their stability or solubility or nutritional properties.

In a particular preferred embodiment of the present invention, the macronutrients are provided in the form of a foodstuff or a fraction thereof, preferably as milk or a protein fraction thereof. Preferred milk proteins or milk protein fractions in accordance with the present invention comprise whey proteins, α-lactalbumin, β-lactoglobulin, bovine serum albumin, casein acid, caseinates, or α, β, κ-casein, for example.

Instead of or in addition to milk proteins also other suitable dietary protein sources may be used, for example animal proteins, such as meat proteins and egg proteins; vegetable proteins, such as soy protein, wheat protein, rice protein, and pea protein; mixtures of free amino acids; or combinations thereof.

If a proteinaceous material is used as macronutrient in the framework of the present invention it may be any composition containing protein material and in particular it may be a solution or dispersion of milk proteins or soy milk proteins: whey proteins, acid whey protein, sweet whey proteins, whey protein concentrates, whey protein isolate, demineralized whey powder or caseinates, for example.

When the proteinaceous material as macronutrient is brought into contact with the at least one enzyme or a functional part thereof exhibiting the enzymatic activity, it is generally preferred if, the protein content varies for example within the range of about 70 to 95% by weight, to achieve an optimal hydrolysis. In general it is preferred if the starting material is as rich in protein as possible.

The proteins present in the proteinaceous material may be modified with proteolytic enzymes obtained from synthetic genes to yield a protein hydrolysate having a degree of hydrolysis (α-amino-N/Nₜₒₜ) of preferably about 10-50%.

During hydrolysis, the concentration of proteinaceous material in solution or in suspension is preferably around 5-20% by weight, and the material could be pasteurised before introducing proteases. The ratio enzyme/protein may be 0.1-10% weight/weight and is preferably about 0.25 to 4%.

Hydrolysis may be conducted at a temperature of about 20° C.-80° C. during 30 minutes to 10 hours, for example of about 35° C. to 65° C., during 30 minutes to 10 hours, preferably 30 min to 4 hours at pH values within the range of 2.5 to 11, for example at pH 4.5, 7.0, 8.0, and 8.5. If desired the pH of the solution can be adjusted and regulated with citric acid, food grade HCl or NaOH, NH₄OH, KOH, Ca(OH)₂ for instance at a concentration of 2N pure or in blend.

Then, the protein hydrolysate may be submitted to a heat treatment for about 0.1 to 10 min at a temperature of about 70 to 110° C. to inactivate residual enzymes (i.e. proteases).

Optionally, the protein hydrolysate solution thus obtained may be clarified by centrifugation and/or ultrafiltration to remove insoluble and intact proteins respectively, and the clear solution recovered. It is possible to use at industrial scale different type of membranes (spiral, tubular, flat, allow fibers) made with different materials (minerals, polysulfone, . . . ) and having different cut off limits between 1.000 and 100.000 Daltons.

The recovered clear hydrolysate solution may, if desired, be concentrated by evaporation to a dry solid content of 10-50% for a subsequent treatment or spray dried.

The protein hydrolysate solution thus obtained may further be submitted to precipitation treatment by solvent, acid, or salts, for example, followed by a centrifugation. In the precipitation treatment, concentration of hydrolysate solution increases the yield and reduces the quantities of solvent. For example, ethanol may be added to obtain a final concentration within 15-60% volume/volume at a temperature of about 4° C. to 25° C. After one hour of incubation, a centrifugation (30 min at 4500 g) may allow to separate soluble and insoluble peptides. Depending on the proteolysate one can use acid (phosphoric or chlorhydric, for example) or phospho-calcic precipitation. Then, solvents can be removed by evaporation and salts by electrodialysis for instance.

The foodstuff of the present invention may be a food product intended for human consumption, an animal food product or a pharmaceutical composition. For example, it may be a nutritional composition, a nutraceutical, a drink, a food additive or a medicament. In a particular preferred embodiment of the present invention the foodstuff may be an infant formula.

The foodstuff of the present invention may also be an ingredient used in one of the foodstuffs listed above.

The enzyme or the functional part thereof may be obtained from the synthetic gene by any means that are known in the art. For example, the synthetic gene coding for the enzyme or the functional part thereof, may be cloned into a cell, such as a micro-organism, for example a yeast cell, fungal cell or a bacterial cell; an insect cell or a mammalian cell to ensure proper protein expression. Alternatively, the enzyme may also be produced in a cell free expression system.

The synthetic gene may be cloned into the micro-organism and/or used in a cell free expression system in an expression cassette, comprising the synthetic gene and at least one regulatory control sequence.

If the synthetic gene is cloned into a cell, for example a micro-organism, this may be carried out by means of transformation of the micro-organism with an expression vector that comprises the synthetic gene. Alternatively, the synthetic gene may also be incorporated into the genome of the cell.

If a micro-organism is used for the purposes of the present invention, it is in particular preferred, if the micro-organism used is a food grade micro-organism. “Food grade” means a material that is approved for human or animal consumption. Food grade micro-organisms have the advantage that they can be added as a culture or as a fraction of a culture to the food product with macronutrients to be modulated and that they do not have to be removed afterwards from the food product.

The enzyme or the functional part thereof to be obtained from the synthetic gene should be selected based on the intended modulation of the macronutrient. In so far the nature of the enzyme or the functional part thereof to be used is not particularly limited in the framework of the present invention.

It is however preferred if the synthetic gene coding for the enzyme or the functional part thereof is a synthetic gene based on the sequence of the porcine, bovine or human mRNA or on the sequence of the porcine, bovine or human enzyme.

Preferably, the at least one enzyme is selected from the group consisting of oxidoreductases, transferases, hydrolases, lyases, isomerases, ligases, or precursors thereof.

If it is intended to digest a macronutrient, it is preferred if the enzyme is a hydrolase.

Preferred hydrolases are
those that cleave ester bonds, for example esterases, e.g., nucleases, phosphodiesterases, lipases, phosphatases;
those that cleave sugars, for example glycosylases/DNA glycosylases, glycoside hydrolases
those that cleave ether bonds;
those that cleave peptide bonds, for example proteases or peptidases
those that cleave carbon-nitrogen bonds other than peptide bonds
those that cleave acid anhydrides, for example acid anhydride hydrolases, including helicases and GTPase
those that cleave carbon-carbon bonds
those that cleave halide bonds
those that cleave phosphorus-nitrogen bonds
those that cleave sulfur-nitrogen bonds
those that cleave carbon-phosphorus bonds
those that cleave sulfur-sulfur bonds; and or
those that cleave carbon-sulfur bonds.

The hydrolase may be selected from the group consisting of nucleases, endonucleases, exonucleases, acid hydrolases, phospholipase A, acetylcholinesterase, cholinesterase, lipoprotein lipase, Ubiquitin carboxy-terminal hydrolase L1, Alkaline phosphatase, Fructose bisphosphatase, Phospholipase C, CGMP specific phosphodiesterase type 5, Phospholipase D, Restriction enzyme Type 1, Deoxyribonuclease I, RNase H, Ribonuclease, Amylase, Sucrase, Chitinase, Lysozyme, Maltase, Lactase, Beta-galactosidase, Hyaluronidase, Alanine aminopeptidase, Angiotensin-converting enzyme, proteases, serine proteinases, Chymotrypsin, Trypsin, Thrombin, Factor X, Plasmin, Acrosin, Factor VII, Factor IX, Factor XI, Elastase, Factor XII, Tissue plasminogen activator, Protein C, Separase, Pepsin, Rennet, Renin, Trypsinogen, Plasmepsin, Matrix metalloproteinases, Metalloendopeptidases, Urease, Beta-lactamase, Arginase, Adenosine deaminase, GTP cyclohydrolase I, Nitrilase, Helicases, DnaB helicase, RecQ helicase, ATPase, NaKATPase, ATP synthase, Kynureninase, carbohydrase, esterase, xylanase, glucanase, mannanase, pectinase or combination thereof.

If the macronutrient is a protein or a protein source, for example a milk protein fraction, the macronutrient, for example the milk protein fraction, may be modulated by digesting the milk protein fraction with at least one proteinase or a functional part thereof obtained from a synthetic gene. Any proteinase may be used for this purpose. For example, serine proteases, threonine proteases, cysteine proteases, aspartic acid proteases, metalloproteases, glutamic acid proteases, or mixtures thereof may be used. In particular preferred are trypsin and/or chymotrypsin obtained from a synthetic gene with the protein sequence of porcine trypsin and/or chymotrypsin.

The synthetic genes coding for the enzymes or the functional part thereof used in the present invention typically contain the same gene sequence as the gene in its natural form. However the gene sequence may also be altered, for example to optimize the synthetic gene for codon usage of the expressing micro-organism.

It should be understood that the synthetic genes may also code for a precursor of the enzyme or the functional part thereof that is intended for use in the framework of the present invention.

For the purpose of the present invention the term “enzyme or a functional part thereof capable of modulating macronutrients” shall include precursors of such enzymes or functional parts thereof.

Precursors may be enzymatically inactive and might require activation before they exhibit their enzymatic activity. The expression of precursors has the advantage that enzymes that might be a threat to the expressing cell can be safely expressed as precursors without any risk for the cell.

For example, proteinases are typically expressed in their zymogen form, a precursor of the active proteinase. This zymogen requires activation to arrive at an active proteinase. Trypsinogen is the zymogen form of trypsin and chymotrypsinogen is the zymogen form of chymotrypsin. Both, chymotrypsinogen and/or trypsinogen may be expressed for the purposes of the present invention

An activation of the zymogen may include a biochemical change, such as a hydrolysis reaction revealing the active site, or changing the configuration to reveal the active site, for the zymogen to become an active enzyme.

In the framework of the present invention activation of the precursor, for example the zymogen, may also occur by treating the zymogen form with a proteinase to generate the active enzyme. Oftentimes, the zymogens may also perform an autolytic reaction to activate themselves, so that an extra activation step may be omitted. Further, the zymogen form may also be activated by residual proteinases that are already present in the food product to be treated with the enzyme or the active fraction thereof. Further ways to activate zymogen forms of enzymes are well within the knowledge of those of skill in the art and do not need to be exemplified here.

It is preferred if the synthetic gene shares at least 75%, preferably at least 80%, more preferably at least 90%, even more preferred at least 95%, most preferred at least 99% DNA-sequence identity with the natural gene.

It is equally preferred, if the enzyme or the functional part thereof obtained from the synthetic gene shares at least 75%, preferably at least 80%, more preferably at least 90%, even more preferred at least 95%, most preferred at least 99% and ideally 100% protein sequence identity with the natural enzyme.

It is also preferred, if the enzyme or the functional part thereof obtained from the synthetic gene exhibits at least 75%, preferably at least 80%, more preferably at least 90%, even more preferred at least 95%, most preferred at least 99% and ideally at least 100% of the activity of the natural enzyme.

The synthetic gene coding for the enzyme or the functional part thereof may be obtained by any method that is known in the art.

Synthetic genes are commercially available from numerous sources. A Google search for “gene-synthesis” yields 85900 hits.

For example, the synthetic gene may be obtained by a total gene synthesis, by ligation of preformed duplexes of phosphorylated overlapping oligonucleotides (Scarpulla, R. C. et al., (1982) Anal. Biochem., 121, 356-365; Gupta, N. K., et al., (1968), Proc. Natl. Acad. Sci. USA, 60, 1338-1344), the Fok I method (Mandecki, W. and Bolling, T. J. (1988); the PCR assembly method (Stemmer, W. P., et al., (1995) Gene, 164, 49-53), and/or methods including self-priming PCR (Dillon & Rosen, 1990, Biotechniques 9: 298, 300, dual asymmetrical PCR (DA-PCR) (Sandhu et al., 1992, Biotechniques 12:14-16), PCR-based assembly (Stemmer et al., 1995, Gene 164: 49-53) and the template directed ligation (TDL) (Strizhov et al., 1996, Proc Natl Acad Sci USA 93: 15012-15017) thermodynamically balanced inside-out (TBIO) (Gao et al., 2003, Nucleic Acids Res 31: e143), two-step total gene synthesis coupling with dual asymmetrical PCR and overlap extension PCR (Young & Dong, 2004, Nucleic Acids Res 32: e59), PCR-based two-step DNA synthesis (PTDS) (Xiong et al., 2004a, Nucleic Acids Res 32: e98), successive extension PCR (Xiong et al., 2004, J Biochem Mol Biol 37:282-291) and microchip based technology for multiplex gene synthesis (Tian et al., 2004, Nature 432: 1050-1054), DNA synthesis machines (Pon & Yu, 2004, Nucleic Acids Res 32: 623-631; Pon & Yu, 2005, Nucleic Acids Res 33: 1940-1948), or combinations of these methods.

The present invention also relates to a product comprising a macronutrient modified by an enzyme or a functional part thereof obtained from a synthetic gene.

The product may be a food composition, for example a food product intended for human consumption, an animal food product or a pharmaceutical composition. For example, the product may be a nutritional composition, a nutraceutical, a drink, a food additive, a medicament or a composition with altered sensory properties. It is preferred if the product is an infant feeding formula.

One embodiment of the present invention is a food composition comprising a milk protein fraction hydrolysed by a proteinase, such as for example trypsin and/or chymotrypsin, derived from a synthetic gene, for example with a DNA-sequence that allows to express a protein with the protein sequence of porcine trypsin and/or chymotrypsin.

The product of the present invention may comprise an enzyme or a functional part thereof derived from a synthetic gene and/or a culture of a micro-organism or a fraction thereof, wherein the micro-organism is capable of expressing the enzyme or a functional part thereof.

The fraction of the culture of a micro-organism capable of expressing the enzyme or a functional part thereof may but does not have to include the micro-organism. If the enzyme or the functional part thereof is at least partially secreted into the medium of the culture, it may be sufficient to bring the macronutrient into contact with a part of the medium. Medium and micro-organisms can be easily separated for example by filtration or centrifugation.

If the food product contains milk proteins as macronutrients which were digested using proteinases, for example trypsin and/or chymotrypsin derived from synthetic genes with a DNA-sequence that allows to express a protein with the protein sequence of porcine trypsin and/or chymotrypsin, the resulting peptide profile after digestion will allow for example the production of a hypoallergenic composition.

Consequently, this invention provides a hypoallergenic composition, for example, for the induction of protein tolerance in at risk individuals of protein allergy containing (i) a “non allergenic” extensively hydrolysed proteins basis and/or (ii) a free amino acid basis, said composition comprising as the active ingredient at least one tolerogenic peptide of the allergenic protein.

The term “non-allergenic” basis is to be understood as a nitrogen source containing a well-balanced amino-acids composition. The “non-allergenicity” is defined for milk proteins as residual allergenicity of individual whey proteins not exceeding 1 ppm and as residual allergenicity of total caseins not exceeding 10 ppm.

This food product may also be used to induce oral tolerance. In the present invention, the term tolerance is to be understood as a state of specific immunological unresponsivness. Both humoral (antibodies) and cell mediated (lymphocyte) pathways of the immune response may be suppressed by tolerance induction. A breakdown of oral tolerance is considered to be the underlying cause of food allergy.

The term tolerogenic peptides is to be understood as proteic fragments, corresponding to parts of the native protein, sized from 200 to 6000 Da (3 to 50 amino acids), and preferably between 500 to 3000 Da and being able to induce specific oral tolerance to native proteins.

In a preferred embodiment, said tolerogenic peptides are present in the form of (i) isolated tolerogenic peptidic fractions of hydrolysis of proteinaceous material containing the allergenic protein and/or (ii) synthetically prepared tolerogenic peptides.

Typically, such compositions contain a source of nitrogen which may provide 7 to 25% of the total energy, a source of carbohydrates which may provide at least 28 to 66% of the total energy, a source of lipids which may provide at least 25 to 60% of the total energy and at least one tolerogenic peptide of the different proteins.

A major advantage of this composition is to induce oral tolerance in “at risk” individuals, in order to avoid eventual sensitization by use of native tolerogens.

Individuals, in particular infants, are considered being “at risk” of protein allergy when either one, two parents or one sibling is atopic.

The tolerogenic peptides derived from protein hydrolysis offer both hypoallergenic and tolerogenic properties and induce oral tolerance at the humoral and cellular levels.

For example, the tolerogenic peptides may be from milk origin and particularly from β-Lactoglobulin (β-LG), α-lactalbumin, bovin serum albumin or casein origin.

For the preparation of said composition, for example the following tolerogenic peptides may be used, possibly in the form of a peptidic fraction containing the following peptides: H₂N-I-D-A-L-N-E-N-K-COON, H₂N-V-L-V-L-D-T-D-Y-K,-K-COOH or H₂N-T-P-E-V-D-D-E-A-L-E-K-F-D-K-COOH from β-Lactoglobulin. The composition may also contain tolerogenic peptides from milk origin such asp-lactoglobulin or caseins, for example.

The composition of the present invention, for example a composition intended for individuals at risk of milk protein allergy, may be prepared by hydrolysing a proteinaceous material containing the allergenic protein to a degree of hydrolysis of about 10 to 50% by using at least one proteinase or a functional part thereof obtained from a synthetic gene; inactivation of the enzymatic activity, for example by a heat treatment; clarification of the protein hydrolysate solution; optionally followed by a precipitation treatment. The tolerogenic peptidic fractions may be further purified by chromatography.

For this purpose, the protein hydrolysate solution may be passed into a column filled with adsorption, ion exchange or hydrophobic resin at a flow rate of 0.1-4 column volumes per hour at a temperature of about 4° C. to 60° C. Before the chromatography treatment, the protein hydrolysate can be concentrated to provide a solution having a dry solid content of 8-35% by weight.

During chromatography, a fraction of peptide is absorbed into the resin by passing the hydrolysate solution into a column filled with the convenient support at a rate of 0.1-4 column volumes per hour. It is possible to use at industrial scale the different types of chromatography as: ions exchange, hydrophobic interactions, reverse phases, adsorption (hydroxyapatite, active charcoal, polystyrene base hydrophobic resins . . . ) or covalent chromatography, for example.

In the chromatography treatment, the amount of hydrolysate solution per litre of resin filled column can be as high as 5 litres with the respect to dry solids of 10%.

Preferably, a hydrolysate solution having 20-1000 g of dry solid per litre of resin is passed into the resin filled column. The chromatography treatment may be carried out at a pH of about 2 to 10 preferably 6-8, for the clarified hydrolysate solution. The chromatography treatment can be conducted at a temperature of about 4° C. to 60° C.

For example, the chromatography treatment to select tolerogenic fractions from-lactoglobulin may consist in using
a strong cationic resin equilibrated with 0.1 N HCl at a flow rate of 1 volume/hour. The non-retained fraction may be eluted with 3 volumes of water, the second fraction (fraction containing tolerogenic peptides) may be eluted with 0-0.5 N NaOH, and the third fraction may be eluted with 0.1 NHCl.
a reverse phase (C 18) resin equilibrated with pure water. Non retained fractions are eluted with water, then step by step (20% and 40% of ethanol) the second and the third fraction are recovered.
a strong anionic resin equilibrated with 0.1N NaOH. The non retained fraction may be eluted with 3 volumes of water. The second fraction may be eluted with 0.5N HCl, the third one with 0.1 N NaOH.

The most preferred method is to treat with resin a neutral solution, in that case, no pH adjustment is required-after hydrolysis step and the salt content of the product will be lower.

To conclude the chromatography treatment, the column can be eluted with pure water, then water containing salts, buffer, acids, bases, or organic solvents at a temperature of 4-60° C. Elution is realised step by step or by a gradient of concentration. The solutions that have passed through the column are recovered. If necessary, salts, solvents, acids and/or bases, are removed from the recovered solution, and the recovered solutions can be concentrated to dry solids content of 35-65% and spray dried.

These peptides are then specific fragments corresponding to a part of the native protein sequence or to a part of the specific tryptic peptides of hydrolysed protein.

These tolerogenic peptides can be used for the preparation of a composition inducing oral tolerance to native proteins, said composition is intended for mammals susceptible to protein allergy and particularly human and pets.

This preferred method is well suited for the treatment of hydrolysates prepared from various protein concentrations (Nₜₒₜ%=N*6.38) for modifying the ratio of tolerogenic activity by residual antigenicity from proteinaceous material. If one defines arbitrarily the antigenicity of a native protein to be 10⁶ (as 10⁶ Lg/g of protein), and the tolerogenic response to be 1, then, for a native protein, this ratio is 10⁻⁶. Therefore, the ratio qualifying the tolerogenic activity of one given fraction or tolerogenic peptide should be at least 2×10⁻².

The term allergen is to be understood as a protein or macropeptide capable of initiating allergic reactions in humans, particularly at risk infants or nurslings.

The composition of the present invention may contain tolerogenic peptides in an amount sufficient to induce oral tolerance which is preferably the one which allows a complete oral tolerance induction, namely the one which prevents from any reaction after DBPCFC (double blind placebo controlled food challenge) performed with cow's milk. Accordingly, tolerogenic peptides may be present in an amount of about 0.01% to 10% (nitrogen source of the protein), for example and preferably about 0.1 to 0.2% of total peptides.

Based on the above disclosure, those skilled in the art will understand that the modified macronutrients prepared by the method of the present invention may be used for the production of a product to facilitate absorption and food tolerance, for example in subjects with a compromised functioning of the gastro-intestinal tract and/or in subjects with challenging feeding issues.

Clinical applications include: early post surgical feeding, malabsorption, chronic diarrhoea, hypoalbuminemia, pancreatic insufficiency, short bowel syndrome, HIV/AIDS, Crohn's disease, growth failure, radiation enteritis, cystic fibrosis, and elevated gastric residuals.

The modified macronutrients prepared by the method of the present invention may be used for the production of a product to treat or prevent allergic disorders, in particular food allergies, such as cows' milk allergy, in particular in infants; and/or to induce oral tolerance.

Those skilled in the art will understand that they can freely combine all features of the present invention described herein, without departing from the scope of the invention as disclosed. In particular, features described for the method of the present invention may be applied to the product of the present invention and vice versa.

Further advantages and features of the present invention are apparent from the following sequence listing, examples and figures.

The sequence listing shows

SEQ-ID NO 1: Porcine cationic trypsinogen protein

SEQ-ID NO 2: Anionic trypsinogen protein

SEQ-ID NO 3: Chymotrypsinogen B protein

SEQ-ID NO 4: Chymotrypsinogen C protein

SEQ-ID NO 5: Intein-cationic trypsinogen fusion protein sequence

SEQ-ID NO 6: Intein-anionic trypsinogen fusion protein sequence

SEQ-ID NO 7: Intein-Chymotrypsinogen B fusion protein sequence

SEQ-ID NO 8: Intein-Chymotrypsinogen C fusion protein sequence

SEQ-ID NO 9: Synthetic cationic trypsinogen gene sequence

SEQ-ID NO 10: Synthetic anionic trypsinogen gene sequence

SEQ-ID NO 11: Synthetic Chymotrypsinogen B gene sequence

SEQ-ID NO 12: Synthetic Chymotrypsinogen C gene sequence

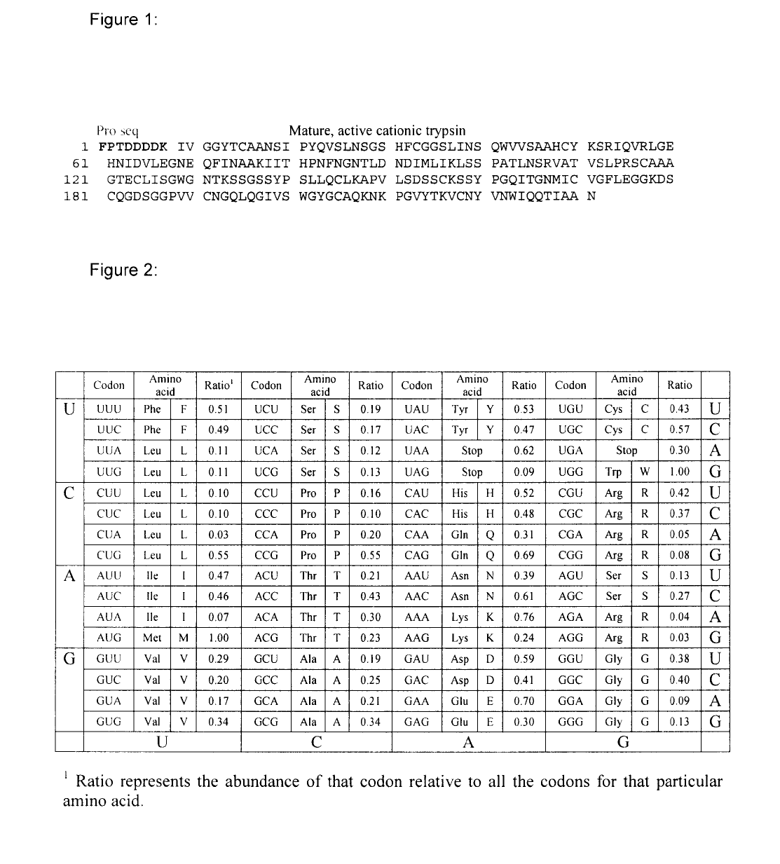

FIG. 1 shows the porcine cationic trypsinogen sequence from P00761 (231 aa).

FIG. 2 shows the codon usage table for Escherichia coli as modified from Maloy, S., V. Stewart, and R. Taylor. 1996. Genetic analysis of pathogenic bacteria. Cold Spring Harbor Laboratory Press, NY.

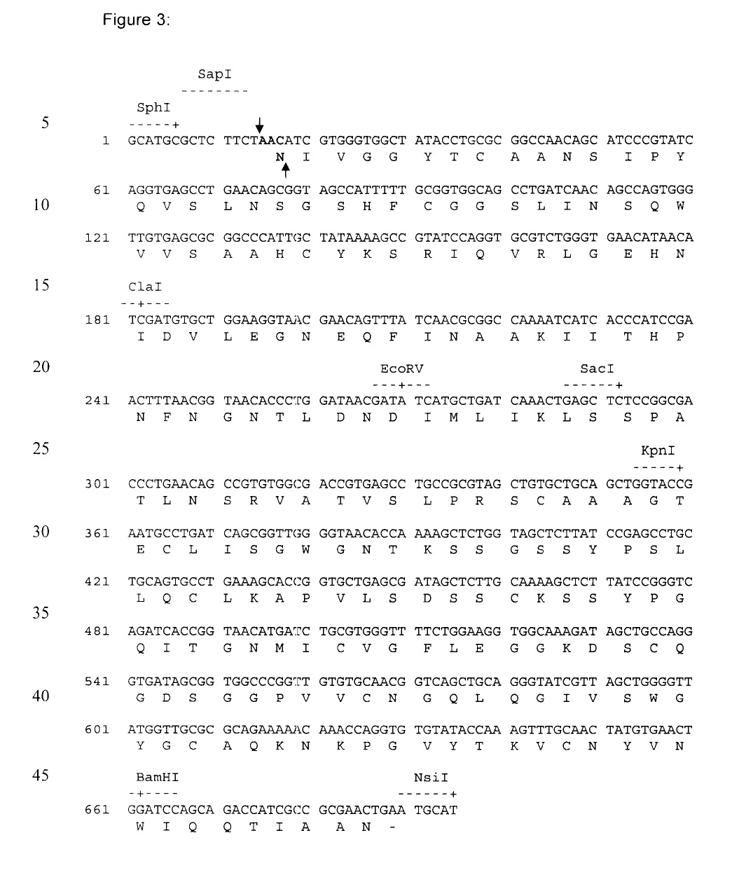

FIG. 3 shows the synthetic cationic trypsinogen gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

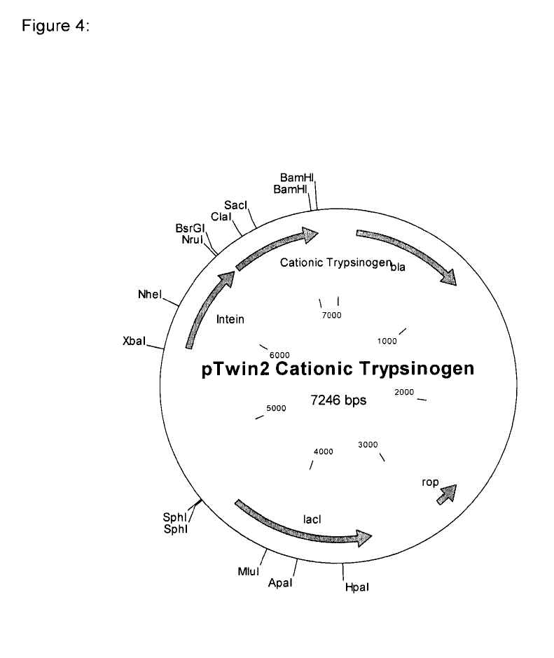

FIG. 4 shows a plasmid map of pTwin2-Cationic-trypsinogen for the expression of the fused intein-trypsin protein.

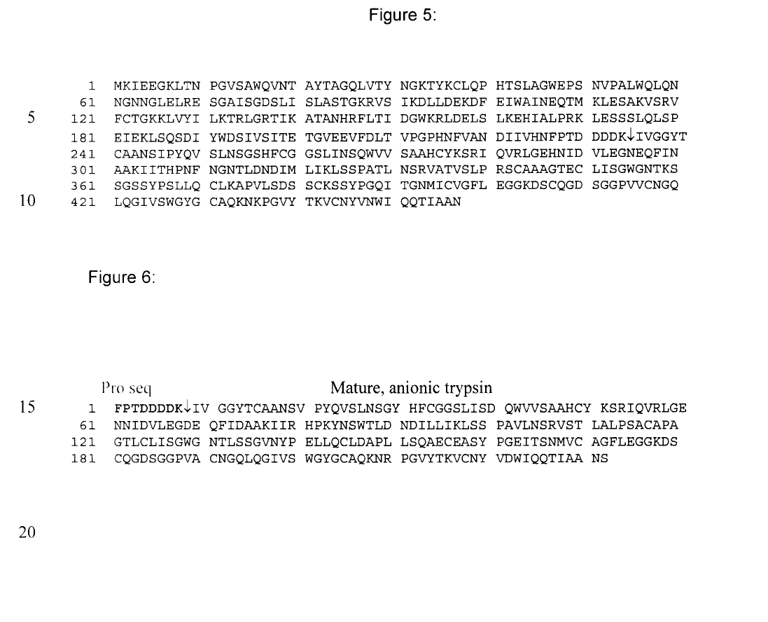

FIG. 5 shows an intein-cationic trypsinogen fusion protein sequence. The intein sequences are shown in red and the porcine trypsinogen in black.

FIG. 6 shows the porcine anionic trypsinogen sequence (232 aa).

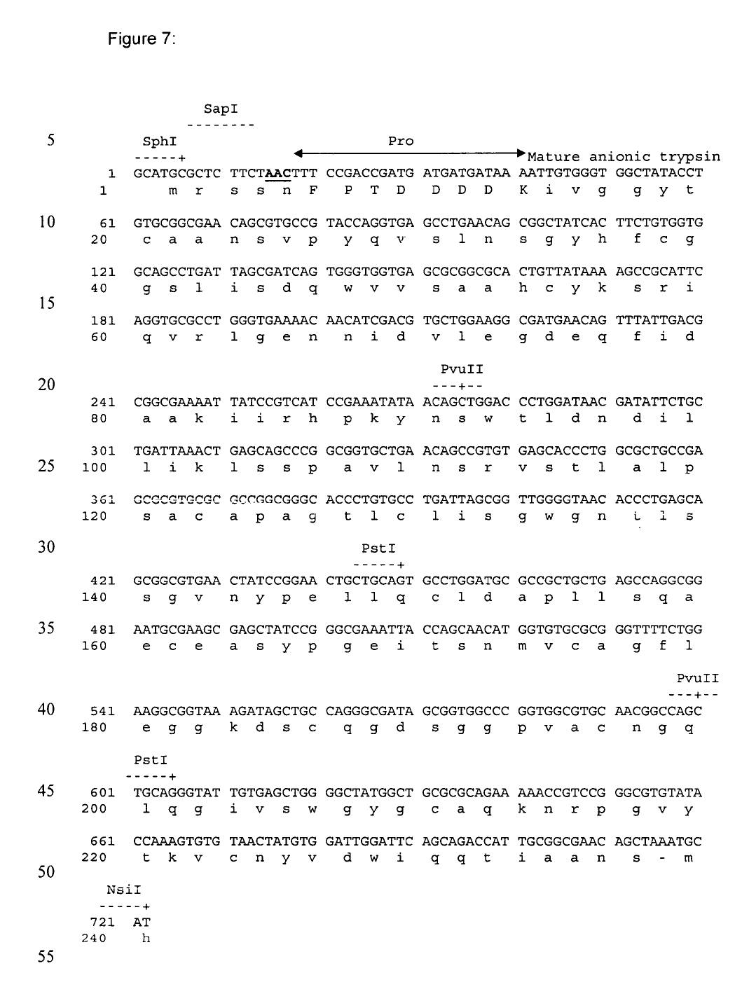

FIG. 7 shows the synthetic anionic trypsinogen gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

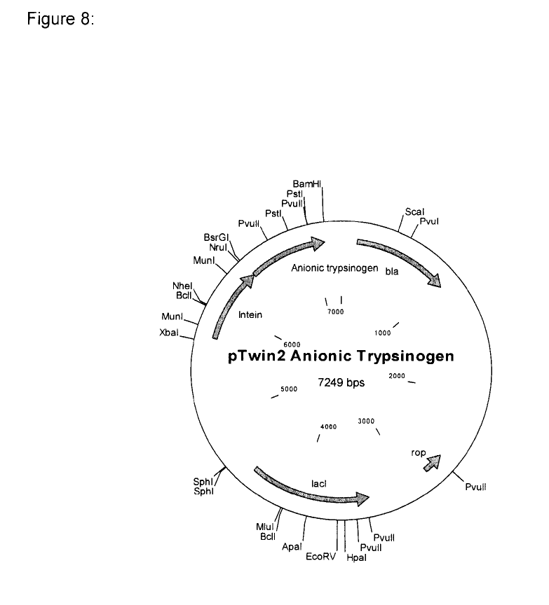

FIG. 8 shows a plasmid map of pTwin2-anionic trypsinogen for the expression of the fused intein-trypsin protein.

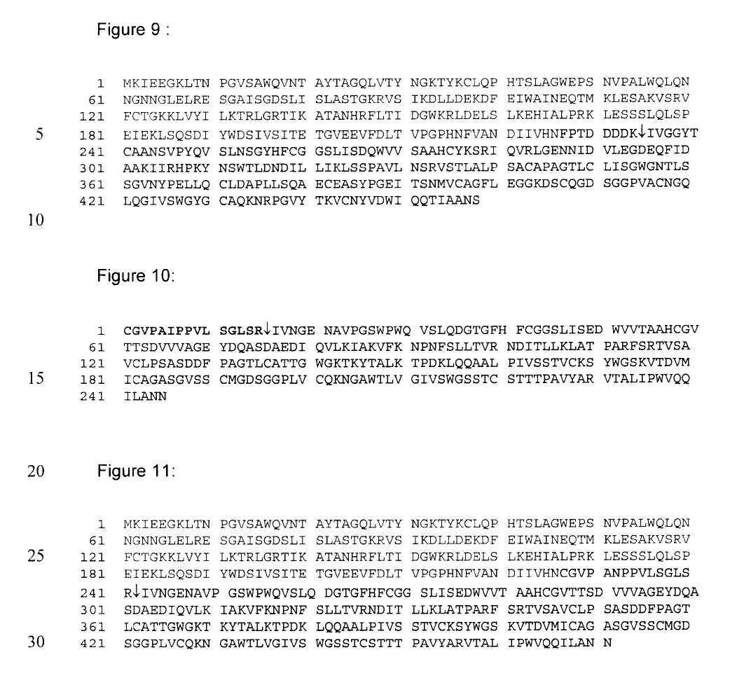

FIG. 9 shows the Intein-anionic trypsinogen fusion protein sequence. The intein sequences are shown in red and the porcine cationic trypsinogen in black.

FIG. 10 shows the chymotrypsinogen B sequence.

FIG. 11 shows an intein-chymotrypsinogen B fusion protein sequence. The intein sequences are shown in red and the porcine chymotrypsinogen B in black.

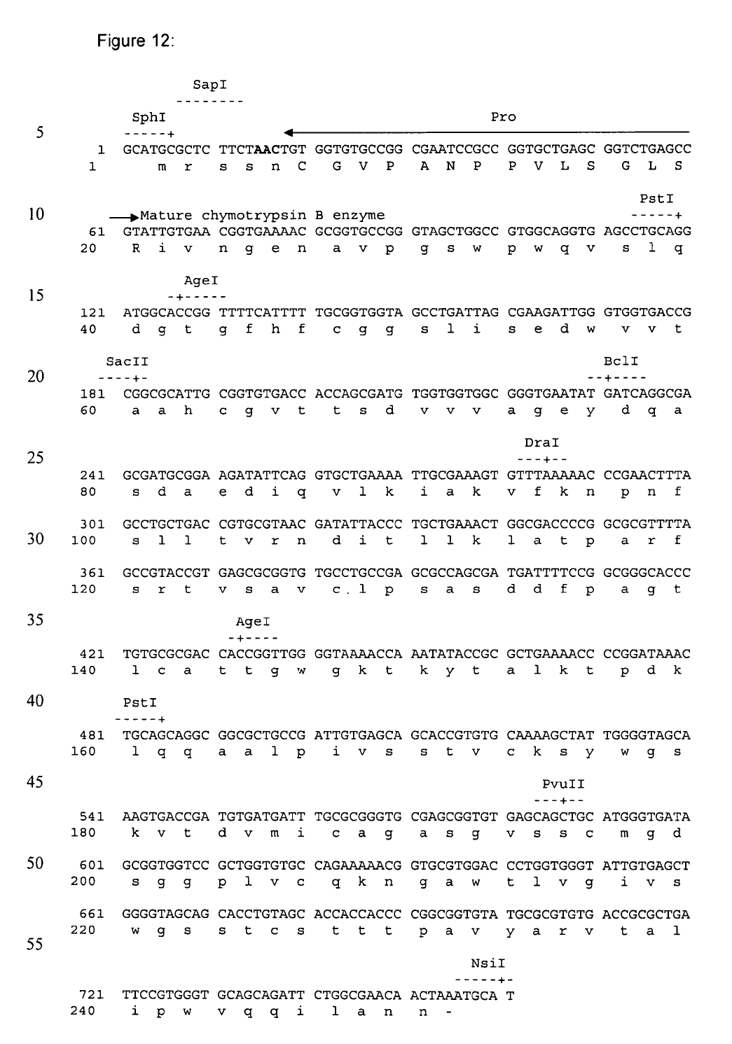

FIG. 12 shows the synthetic chymotrypsinogen B gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

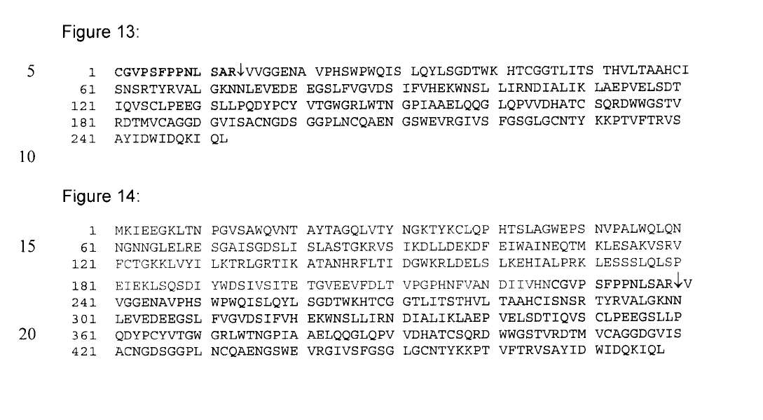

FIG. 13 shows the chymotrypsinogen C sequence.

FIG. 14 shows an intein-chymotrypsinogen C fusion protein sequence. The intein sequences are shown in red and the porcine chymotrypsinogen C in black.

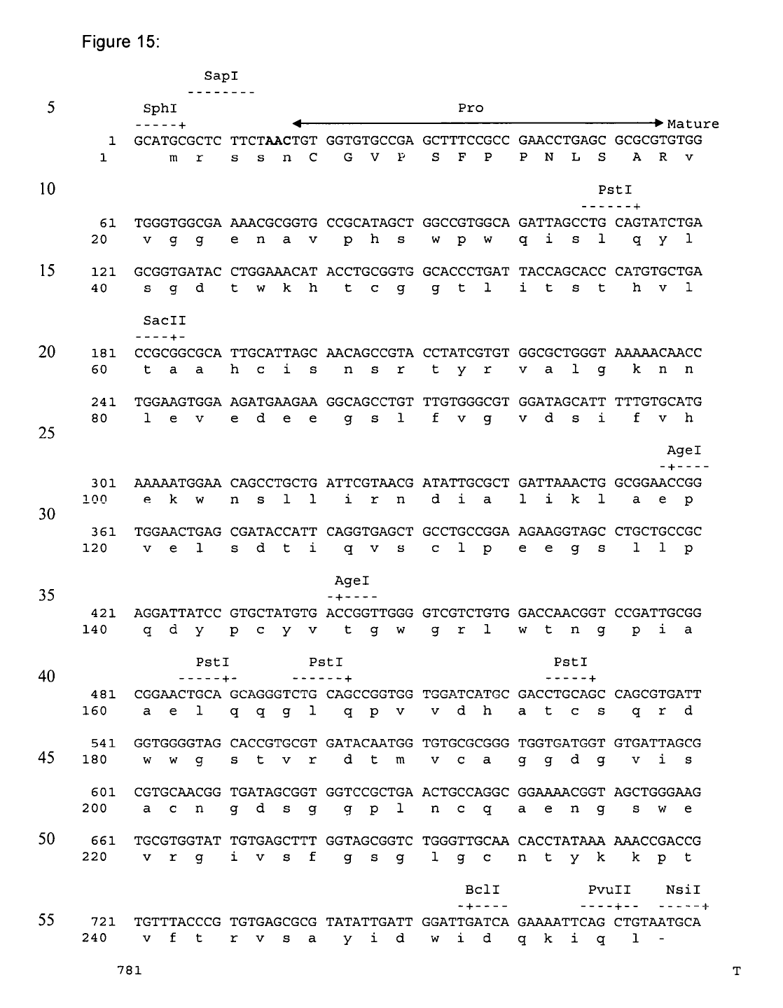

FIG. 15 shows the synthetic chymotrypsinogen C gene sequence. The restriction enzyme SapI cleaves the DNA upstream of its recognition site leaving a 3 base pair overhang (AAC encoding the Asn amino acid marked in red) that reconstitutes the last amino acid of the intein cleavage site.

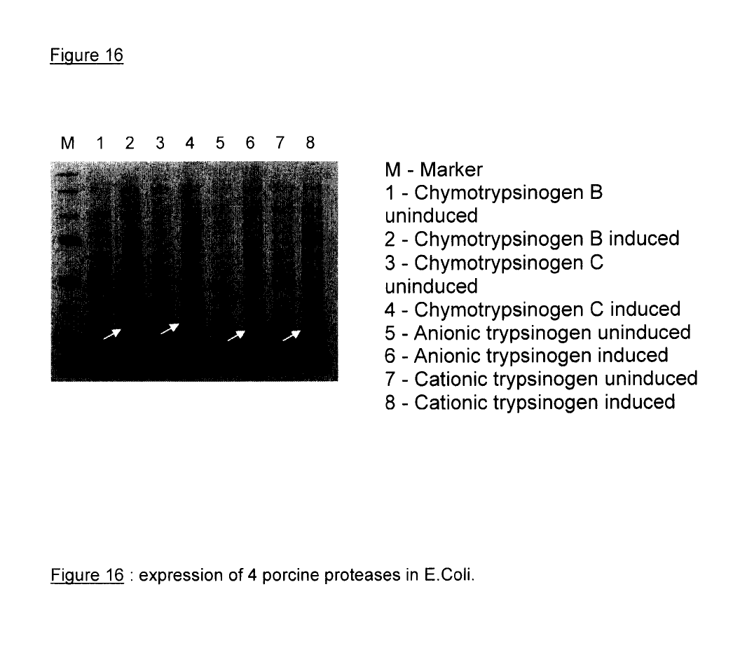

FIG. 16 shows the expression of the 4 porcine proteases in E. coli: Lane 1 shows the insoluble cell wall associated proteins for the chymotrypsinogen B expression strain before induction, while lane 2 shows the same strain after 4 hrs of IPTG induced expression. The chymotrypsinogen B enzyme is indicated by the arrow. The 3 other proteases are as indicated in the paired lanes. The figure indicates the actual expressions of the proteases have been obtained.

### EXAMPLE 1

### Expression of Porcine Cationic Trypsin in Escherichia coli

The 231 amino acid porcine cationic trypsinogen sequence is obtainable from Swissprot file P00761 where the first 8 amino acids constitute the pro sequence that is cleaved of to produce the active enzyme trypsin as shown in FIG. 1.

The mature cationic trypsin protein sequence was translated to DNA sequence using Escherichia coli most frequently used anti codons using the codon usage table shown in FIG. 2.

The gene sequence was also controlled for the accuracy of the protein sequence and the presence of dyad symmetries that could interfere with transcription and the sequence was modified to remove the strongest structures. SphI and NsiI restriction sites were added to the 5′ and 3′ ends, respectively, to allow gene synthesis and cloning. Additionally a SapI restriction site was introduced at the 5′ end of the trypsin gene to allow cloning into plasmid pTwin2 (New England Biolabs). In this construction the cationic trypsinogen sequence is fused to the intein in pTwin2 and which after auto cleavage will release the cationic trypsinogen enzyme. The final sequence is given in FIG. 3.

This gene can be synthesised directly from overlapping oligonucleotides and then cloned into either of the cloning vectors pGEM5 or pGEM7 (Promega) and the DNA sequence may confirmed by DNA sequence analysis. The efficiency of cloning is improved using 3′ overhangs at the extremities due to oligonucleotide synthesis progressing from 3′ to 5′, hence ensuring that the 3′ end is complete (cloning using 5′ overhangs suffers as not all oligonucleotides reach the correct 5′ end). The final plasmid was then digested with the restriction enzymes SapI+NsiI and cloned into pTwin2 digested with SapI+PstI to give the plasmid shown in FIG. 4.

pTwin2 contains a mini-intein derived from the Synechocystis sp dnaB (Wu, H. et al., 1998. Biochim. Biophys. Acta. 1387:422-432) that has been engineered to undergo pH and temperature dependent cleavage at its C-terminus (Mathys, S., et al., 1999, Gene. 231:1-13). Inteins are peptide sequences sometimes found within proteins that are auto-catalytically removed to create the final active enzyme. This allows the purification of enzymes with any amino acid at the amino-terminus and not restricted to methionine.

SapI cleaves in this manner:

5′ . . . GCTCTTCN
3′ . . . CGAGAAGNNNN

The intein-trypsin fusion protein (FIG. 5) may be expressed from this plasmid or transferred into another expression plasmid such as pET24 or one of the numerous expression plasmids for E. coli. Expression may be achieved similar to the method described by Kiraly, O., et al., 2006, Protein Expr. Purif. 48:104-111. The plasmid pTwin2 uses the strong T7 promoter that is inducible by isopropyl 1-thio β D-galactopyranoside (IPTG) in an appropriate host strain such as ER2566 (New England Biolabs). Bacterial cells carrying the plasmid pTwin2-trypsin are cultivated in LB medium containing 100 μg/ml ampicillin for plasmid selection at 37° C. with aeration. At an optical density of approximately 0.5-0.7 OD₆₀₀, IPTG is added to a final concentration of 0.3-0.5 mM and the culture incubated at 15° C. for a further 16 h. Alternative conditions could be 37° C. for 2 h or 30° C. for 6 h depending on the toxicity of the expressed protein. After this time the cells are harvested by centrifugation (may be frozen at −20° C. until use).

The cells are suspended in 0.1 M Tris-HCl (pH 8.0), 5 mM K-EDTA and the cells are disrupted by sonication. The inclusion bodies containing the intein-trypsin fusion protein are then collected by centrifugation at 18,000 g for 5 minutes. The pellet was washed twice with the above buffer and then dissolved in the denaturing buffer containing 4 M guanidine-HCl, 0.1 M Tris-HCl (pH 8.0), 2 mM K-EDTA and 30 mM dithiothreitol at 37° C. for 30 minutes. Denatured proteins are then rapidly diluted 100× by adding refolding buffer (0.9 M guanidine-HCl, 0.1 M Tris-HCl (pH 8.0), 2 mM K-EDTA and 1 mM L-cysteine, 1 mM L-cystine) and are stirred under argon for 5 minutes and are incubated at 4° C. for 16 h. This solution was diluted in an equal volume of 0.4 M NaCl, centrifuged at 20,000 g for 15 minutes and the supernatant was loaded onto an ecotin affinity column. The column was washed with 20 mM Tris-HCl (pH 8.0), 0.2 M NaCl and the intein-trypsin fusion protein eluted with 50 mM Tris-HCl (pH 8.0). Cleavage of the intein from the trypsin can be achieved by incubating the fusion protein at 25° C. in 20 mM HEPES or Tris-HCl (pH 7.0), containing 500 mM NaCl, and 1 mM EDTA for 16 h. The mature trypsin may be further purified from the intein protein using the ecotin affinity column.

Alternatively, the intein cleavage may be done on the ecotin affinity column, washed and the purified subsequently trypsin eluted.

Alternatively, the gene expression could be performed in a strain of E. coli deficient in thioredoxin reductase to create a reducing environment to favour the formation of disulphide bonds and the direct production of an active enzyme without the need to denature and refold the enzyme (Verheyden, G., et al., 2000. J. Chromatogr. B Biomed. Sci. Appl. 737:213-224.).

Alternatively, a hexahistidine-tail could be engineered at the amino terminus to allow affinity purification using a Ni-NTA-agarose column.

Alternatively, the intein could be replaced by yeast ubiquitin, the recombinant protein purified and the ubiquitin removed using the purified yeast YUH1 enzyme.

### EXAMPLES 2-4

Anionic trypsinogen, Chymotrypsinogen B and Chymotrypsin C can be prepared in accordance with what is described above.

For anionic trypsinogen, reference is made to FIGS. 6-9.

For chymotrypsinogen B reference is made to FIGS. 10-12.

For chymotrypsinogen C reference is made to FIGS. 13-15.

### EXAMPLE 5

### Use of the Enzymes of Examples 1 to 4 to Partially Hydrolyse Whey Protein

254.6 kg of demineralised acidic whey powder, 91.3 kg whey protein concentrate obtained by ultrafiltration of sweet whey and 101.4 kg food-grade lactose are dispersed in 800 kg demineralised water at 60° C. The dispersion is placed in a double-walled reactor thermostatically controlled at 55° C. The dispersion has a dry matter content of 30.1% and a pH of 6.4. The pH is increased to 7.8 by addition of a 20% aqueous dispersion of Ca(OH)₂. 1 kg of a mixture of trypsin and chymotrypsin produced as described above (strength 6 AU/g, trypsin:chymotrypsin activity ratio 15:1-20:1 in USP) dispersed in a 0.01M aqueous solution of HCl is then added at 5 to 10° C. to initiate the hydrolysis. If zymogen forms of trypsin and/or chymotrypsin are used, these may be activated by the addition of proteinases, as it is well known to those of skill in the art. The initial rapid fall in pH is then stopped, the pH being maintained at 7.3 using a pH-stat by automatic compensation with a 2N aqueous KOH solution.

Hydrolysis is continued for 3 hours at 55° C./pH 7.3 after which the pH is increased to 7.6 by adjustment of the pH-stat to the new value. The hydrolysate is passed through a plate-type hear exchanger where it is rapidly heated to 90° C., then to a dwell tube (flow rate 7.5 l/minute, tube volume 40 lg, residence time 5 minutes) and then into a second plate-type heat exchanger where it is cooled to 55° C. The coiled hydrolysate is pumped at a rate of 7.5 l/minute through a T valve into a dwell tube 0.025 m in diameter for a volume of 150 l which corresponds to a residence time of 20 minutes over the entire length of the tube. A further 1 kg of the mixture of trypsin and chymotrypsin is pumped into the hydrolysate stream through the T valve at the entrance to the dwell tube at a rate of 6 l/hour. After pre-heating to 80° C. with a dwell time of 5 minutes the hydrolysate (which has undergone a total dwell time of 20 minutes) is pumped into a UHT steriliser where it is heated to 125° C. over a period of 2 minutes. After cooling, the hydrolysate is spray dried. The powder thus obtained comprises, by weight, 23% peptides, 68% lactose, 4% ash, 2% fats and 3% moisture. The degree of hydrolysis calculated as nitrogen×100/total nitrogen (Nt) is 185 and Nt is 3.56%.

Analysis by SDS-PAGE confirms the absence of protein bands. In particular, no bands corresponding to bovine serum albumin, alpha-lactalbumin, beta-lactoglobulin or the H and L chains of IgG are observed.

### EXAMPLE 6

### Preparation of Infant Formula Using the Partial Whey Hydrolysate of Example 5

The procedure of Example 5 is followed up to completion of the second hydrolysis. The hydrolysate is passed to a thermostatically controlled tank and held at 60° C. during the addition of an equivalent quantity of a solution of maltodextrin and starch having a dry matter content of 50% with mineral salts dissolved in demineralised water. The mixture is heated to 75° C. in a plate-type heat exchanger. A mixture of palm olein, coconut oil, safflower oil, lecithin and fat soluble vitamins is melted at 65° C. and added to the hydrolysate mixture in a quantity corresponding to 10% of the hydrolysate mixture. The complete mixture is pre-heated to 80° C. for 5 minutes and then to 125° C. for 2 minutes by direct injection of steam. The heat-treated mixture is cooled to 70° C. in an expansion vessel, homogenised in two stages first at 20 MPa and then at 5 MPa and cooled to 10° C. first in a plate-type heat exchanger and then in an intermediate storage tank. Then, a 10% solution of citric acid in demineralised water, water-soluble vitamins, oligo-elements ands taurine are added. Finally, the mixture is heated to 75° C., homogenised in one pass at 65-170 bar and spray dried. The resulting powder comprises by weight 12.5% peptides, 26% fats, 56.2% carbohydrates, 23% minerals and 3% moisture with traces of vitamins and oligo-elements.

1. Method for modulating a macronutrient comprising the steps of
producing at least one synthetic gene coding for at least one enzyme or a functional part thereof capable of modulating macronutrients,
expressing the enzyme or a functional part thereof, and
bringing the macronutrients into contact with the enzyme or a functional part thereof.

2. Method in accordance with claim 1 comprising the steps of
cloning the synthesized gene into a micro-organism capable of expressing this gene,
cultivating the micro-organism in a culture and expressing the enzyme or a functional part thereof, and
bringing the macronutrient into contact with the culture of the micro-organism or a fraction thereof exhibiting the enzymatic activity.

3. Method in accordance with claim 1, wherein the macronutrients are selected from the group consisting of carbohydrates, proteins, and fats.

4. Method in accordance with claim 1, wherein the macronutrients comprise a milk protein fraction that is modulated by digesting the milk protein fraction with at least one proteinase obtained from a synthetic gene.

5. Method in accordance with claim 1, wherein the synthetic gene is cloned into the micro-organism by means of transformation of the micro-organism with an expression vector that comprises the synthetic gene.

6. Method in accordance with claim 1, wherein the micro-organism is a food grade micro-organism.

7. Method in accordance with claim 1, wherein the enzyme is selected from the group consisting of oxidoreductases, transferases, hydrolases, lyases, isomerases, ligases, and precursors thereof.

8. Method in accordance with claim 4, wherein the hydrolase is selected from the group consisting of hydrolases:
that cleave ester bonds;
that cleave sugars;
that cleave ether bonds;
that cleave peptide bonds;
that cleave carbon-nitrogen bonds other than peptide bonds;
that cleave acid anhydrides;
that cleave carbon-carbon bonds;
that cleave halide bonds;
that cleave phosphorus-nitrogen bonds;
that cleave sulfur-nitrogen bonds;
that cleave carbon-phosphorus bonds;
that cleave sulfur-sulfur bonds; and
that cleave carbon-sulfur bonds.

9. Method in accordance with claim 1, wherein the gene sequence is optimized.

10. Method in accordance with claim 1, wherein the synthetic gene coding for the enzyme or the functional part thereof is a synthetic gene based on the porcine, bovine or human gene.

11. Method in accordance with claim 1, wherein synthetic gene is cloned into the micro-organism in an expression cassette, comprising the synthetic gene and at least one regulatory control sequence.

12. Method in accordance with claim 1, wherein the functional part of the enzyme has at least 80% of the activity of the natural enzyme.

13. Method in accordance with claim 1, wherein the gene is obtained by a method selected from the group consisting of total gene synthesis; ligation of preformed duplexes of phosphorylated overlapping oligonucleotides; the Fok I method; the PCR assembly method; self-priming PCR; dual asymmetrical PCR (DA-PCR); PCR based assembly; the template directed ligation (TDL); thermodynamically balanced inside-out (TBIO); two-step total gene synthesis coupling with dual asymmetrical PCR and overlap extension PCR; PCR-based two-step DNA synthesis; successive extension PCR; microchipbased technology for multiplex gene synthesis; and DNA synthesis machines.

14. Product comprising a macronutrient modified by an enzyme or a functional part thereof, wherein the enzyme or a functional part thereof is obtained from a synthetic gene.

15. Product in accordance with claim 14, wherein the product is a food composition comprising a milk protein fraction hydrolysed by trypsin and/or chymotrypsin, derived from a synthetic gene with the DNA-sequence of porcine trypsin and/or chymotrypsin.

16. Method for modulating a macronutrient comprising the steps of
producing a synthetic gene coding for an enzyme or a functional part thereof capable of modulating macronutrients,
expressing the enzyme or a functional part thereof,
activating the enzyme or a functional part thereof so that it exhibits enzymatic activity, and
bringing the macronutrients into contact with the enzyme or a functional part thereof exhibit the enzymatic activity.

17. Method in accordance with claim 1, wherein the macronutrients are provided in the form of a foodstuff or a fraction thereof.


SEQUENCE LISTING (12 sequences)

SEQ ID NO: 1 - protein, 231 aa, suidae
FPTDDDDKIVGGYTCAANSIPYQVSLNSGSHFCGGSLINSQWVVSAAHCYKSRIQVRLGEHNIDVLEGNEQFINAAKIITHPNFNGNTLDNDIMLIKLSSPATLNSRVATVSLPRSCAAAGTECLISGWGNTKSSGSSYPSLLQCLKAPVLSDSSCKSSYPGQITGNMICVGFLEGGKDSCQGDSGGPVVCNGQLQGIVSWGYGCAQKNKPGVYTKVCNYVNWIQQTIAAN

SEQ ID NO: 2 - protein, 232 aa, suidae
FPTDDDDKIVGGYTCAANSVPYQVSLNSGYHFCGGSLISDQWVVSAAHCYKSRIQVRLGENNIDVLEGDEQFIDAAKIIRHPKYNSWTLDNDILLIKLSSPAVLNSRVSTLALPSACAPAGTLCLISGWGNTLSSGVNYPELLQCLDAPLLSQAECEASYPGEITSNMVCAGFLEGGKDSCQGDSGGPVACNGQLQGIVSWGYGCAQKNRPGVYTKVCNYVDWIQQTIAANS

SEQ ID NO: 3 - protein, 245 aa, suidae
CGVPAIPPVLSGLSRIVNGENAVPGSWPWQVSLQDGTGFHFCGGSLISEDWVVTAAHCGVTTSDVVVAGEYDQASDAEDIQVLKIAKVFKNPNFSLLTVRNDITLLKLATPARFSRTVSAVCLPSASDDFPAGTLCATTGWGKTKYTALKTPDKLQQAALPIVSSTVCKSYWGSKVTDVMICAGASGVSSCMGDSGGPLVCQKNGAWTLVGIVSWGSSTCSTTTPAVYARVTALIPWVQQILANN

SEQ ID NO: 4 - protein, 252 aa, suidae
CGVPSFPPNLSARVVGGENAVPHSWPWQISLQYLSGDTWKHTCGGTLITSTHVLTAAHCISNSRTYRVALGKNNLEVEDEEGSLFVGVDSIFVHEKWNSLLIRNDIALIKLAEPVELSDTIQVSCLPEEGSLLPQDYPCYVTGWGRLWTNGPIAAELQQGLQPVVDHATCSQRDWWGSTVRDTMVCAGGDGVISACNGDSGGPLNCQAENGSWEVRGIVSFGSGLGCNTYKKPTVFTRVSAYIDWIDQKIQL

SEQ ID NO: 5 - protein, 458 aa, Artificial Sequence
  Synthesized Fusion protein of Intein and cationic trypsinogen derived from a synthetic gene
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNFPTDDDDKIVGGYTCAANSVPYQVSLNSGYHFCGGSLISDQWVVSAAHCYKSRIQVRLGENNIDVLEGDEQFIDAAKIIRHPKYNSWTLDNDILLIKLSSPAVLNSRVSTLALPSACAPAGTLCLISGWGNTLSSGVNYPELLQCLDAPLLSQAECEASYPGEITSNMVCAGFLEGGKDSCQGDSGGPVACNGQLQGIVSWGYGCAQKNRPGVYTKVCNYVDWIQQTIAANS

SEQ ID NO: 6 - protein, 457 aa, Artificial Sequence
  Synthesized Fusion protein of Intein and anionic trypsinogen derived from a s ynthetic gene
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNFPTDDDDKIVGGYTCAANSIPYQVSLNSGSHFCGGSLINSQWVVSAAHCYKSRIQVRLGEHNIDVLEGNEQFINAAKIITHPNFNGNTLDNDIMLIKLSSPATLNSRVATVSLPRSCAAAGTECLISGWGNTKSSGSSYPSLLQCLKAPVLSDSSCKSSYPGQITGNMICVGFLEGGKDSCQGDSGGPVVCNGQLQGIVSWGYGCAQKNKPGVYTKVCNYVNWIQQTIAAN

SEQ ID NO: 7 - protein, 471 aa, Artificial Sequence
  Synthesized Fusion protein of Intein and chymotrypsinogen B derived from a sy nthetic gene
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNCGVPANPPVLSGLSRIVNGENAVPGSWPWQVSLQDGTGFHFCGGSLISEDWVVTAAHCGVTTSDVVVAGEYDQASDAEDIQVLKIAKVFKNPNFSLLTVRNDITLLKLATPARFSRTVSAVCLPSASDDFPAGTLCATTGWGKTKYTALKTPDKLQQAALPIVSSTVCKSYWGSKVTDVMICAGASGVSSCMGDSGGPLVCQKNGAWTLVGIVSWGSSTCSTTTPAVYARVTALIPWVQQILANN

SEQ ID NO: 8 - protein, 478 aa, Artificial Sequence
  Synthesized Fusion protein of Intein and chymotrypsinogen C derived from a sy nthetic gene
MKIEEGKLTNPGVSAWQVNTAYTAGQLVTYNGKTYKCLQPHTSLAGWEPSNVPALWQLQNNGNNGLELRESGAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLSPEIEKLSQSDIYWDSIVSITETGVEEVFDLTVPGPHNFVANDIIVHNCGVPSFPPNLSARVVGGENAVPHSWPWQISLQYLSGDTWKHTCGGTLITSTHVLTAAHCISNSRTYRVALGKNNLEVEDEEGSLFVGVDSIFVHEKWNSLLIRNDIALIKLAEPVELSDTIQVSCLPEEGSLLPQDYPCYVTGWGRLWTNGPIAAELQQGLQPVVDHATCSQRDWWGSTVRDTMVCAGGDGVISACNGDSGGPLNCQAENGSWEVRGIVSFGSGLGCNTYKKPTVFTRVSAYIDWIDQKIQL

SEQ ID NO: 9 - DNA, 722 bp, Artificial Sequence
  Synthesized Synthetic gene to express cationic trypsinogen
gcatgcgctcttctaactttccgaccgatgatgatgataaaattgtgggtggctatacctgtgcggcgaacagcgtgccgtaccaggtgagcctgaacagcggctatcacttctgtggtggcagcctgattagcgatcagtgggtggtgagcgcggcgcactgttataaaagccgcattcaggtgcgcctgggtgaaaacaacatcgacgtgctggaaggcgatgaacagtttattgacgcggcgaaaattatccgtcatccgaaatataacagctggaccctggataacgatattctgctgattaaactgagcagcccggcggtgctgaacagccgtgtgagcaccctggcgctgccgagcgcgtgcgcgccggcgggcaccctgtgcctgattagcggttggggtaacaccctgagcagcggcgtgaactatccggaactgctgcagtgcctggatgcgccgctgctgagccaggcggaatgcgaagcgagctatccgggcgaaattaccagcaacatggtgtgcgcgggttttctggaaggcggtaaagatagctgccagggcgatagcggtggcccggtggcgtgcaacggccagctgcagggtattgtgagctggggctatggctgcgcgcagaaaaaccgtccgggcgtgtataccaaagtgtgtaactatgtggattggattcagcagaccattgcggcgaacagctaaatgcat

SEQ ID NO: 10 - DNA, 719 bp, Artificial Sequence
  Synthesized Synthetic gene to express anionic trypsinogen
gcatgcgctcttctaactttccgaccgatgatgatgataaaatcgtgggtggctatacctgcgcggccaacagcatcccgtatcaggtgagcctgaacagcggtagccatttttgcggtggcagcctgatcaacagccagtgggttgtgagcgcggcccattgctataaaagccgtatccaggtgcgtctgggtgaacataacatcgatgtgctggaaggtaacgaacagtttatcaacgcggccaaaatcatcacccatccgaactttaacggtaacaccctggataacgatatcatgctgatcaaactgagctctccggcgaccctgaacagccgtgtggcgaccgtgagcctgccgcgtagctgtgctgcagctggcaccgaatgcctgatcagcggttggggtaacaccaaaagctctggtagctcttatccgagcctgctgcagtgcctgaaagcaccggtgctgagcgatagctcttgcaaaagctcttatccgggtcagatcaccggtaacatgatctgcgtgggttttctggaaggtggcaaagatagctgccagggtgatagcggtggcccggttgtgtgcaacggtcagctgcagggtatcgttagctggggttatggttgcgcgcagaaaaacaaaccaggtgtgtataccaaagtttgcaactatgtgaactggatccagcagaccatcgccgcgaactgaatgcat

SEQ ID NO: 11 - DNA, 761 bp, Artificial Sequence
  Synthesized Synthetic gene to express chymotrypsinogen B
gcatgcgctcttctaactgtggtgtgccggcgaatccgccggtgctgagcggtctgagccgtattgtgaacggtgaaaacgcggtgccgggtagctggccgtggcaggtgagcctgcaggatggcaccggttttcatttttgcggtggtagcctgattagcgaagattgggtggtgaccgcggcgcattgcggtgtgaccaccagcgatgtggtggtggcgggtgaatatgatcaggcgagcgatgcggaagatattcaggtgctgaaaattgcgaaagtgtttaaaaacccgaactttagcctgctgaccgtgcgtaacgatattaccctgctgaaactggcgaccccggcgcgttttagccgtaccgtgagcgcggtgtgcctgccgagcgccagcgatgattttccggcgggcaccctgtgcgcgaccaccggttggggtaaaaccaaatataccgcgctgaaaaccccggataaactgcagcaggcggcgctgccgattgtgagcagcaccgtgtgcaaaagctattggggtagcaaagtgaccgatgtgatgatttgcgcgggtgcgagcggtgtgagcagctgcatgggtgatagcggtggtccgctggtgtgccagaaaaacggtgcgtggaccctggtgggtattgtgagctggggtagcagcacctgtagcaccaccaccccggcggtgtatgcgcgtgtgaccgcgctgattccgtgggtgcagcagattctggcgaacaactaaatgcat

SEQ ID NO: 12 - DNA, 781 bp, Artificial Sequence
  Synthesized Synthetic gene to express chymotrypsinogen C
gcatgcgctcttctaactgtggtgtgccgagctttccgccgaacctgagcgcgcgtgtggtgggtggcgaaaacgcggtgccgcatagctggccgtggcagattagcctgcagtatctgagcggtgatacctggaaacatacctgcggtggcaccctgattaccagcacccatgtgctgaccgcggcgcattgcattagcaacagccgtacctatcgtgtggcgctgggtaaaaacaacctggaagtggaagatgaagaaggcagcctgtttgtgggcgtggatagcatttttgtgcatgaaaaatggaacagcctgctgattcgtaacgatattgcgctgattaaactggcggaaccggtggaactgagcgataccattcaggtgagctgcctgccggaagaaggtagcctgctgccgcaggattatccgtgctatgtgaccggttggggtcgtctgtggaccaacggtccgattgcggcggaactgcagcagggtctgcagccggtggtggatcatgcgacctgcagccagcgtgattggtggggtagcaccgtgcgtgatacaatggtgtgcgcgggtggtgatggtgtgattagcgcgtgcaacggtgatagcggtggtccgctgaactgccaggcggaaaacggtagctgggaagtgcgtggtattgtgagctttggtagcggtctgggttgcaacacctataaaaaaccgaccgtgtttacccgtgtgagcgcgtatattgattggattgatcagaaaattcagctgtaatgcat